(19) United States
(12) Patent Application Publication
(10) Pub. No.: US 2007/0099237 A1
(43) Pub. Date: May 3, 2007

(54) Reaction co-crystallization of molecular complexes or co-crystals

(75) Inventor: Nair Rodriguez-Hornedo, Ann Arbor, MI (US)

Correspondence Address: HARNESS, DICKEY & PIERCE, P.L.C., P.O. BOX 828, BLOOMFIELD HILLS, MI, 48303, US

(73) Assignee: The Regents of The University of Michigan

(21) Appl. No.: 11/262,995

(22) Filed: Oct. 31, 2005

(51) Int. Cl. C40B 40/04; A61K 31/55; A61K 31/522; A61K 31/496; A61K 31/192; A61K 31/655

(52) U.S. Cl. 435/7.1; 514/217; 514/165; 514/263.31; 514/254.07; 514/156; 514/569; 514/571

(57) ABSTRACT

Multi-component crystals (co-crystals) are prepared by combining co-crystal components in non-stoichiometric concentrations in solution. The solubility of the molecular complex in the solvent is reduced, increasing the probability that the molecular complex is the least soluble form in the system, upon which it precipitates. A crystalline product is produced without the need for grinding, solvent evaporation, or temperature variation.

### FIELD OF THE INVENTION

This invention relates to reaction co-crystallization of molecular complexes or co-crystals. In particular, the invention relates to methods for preparing and screening co-crystals.

### BACKGROUND OF THE INVENTION

Co-crystallization is an essential processing step in the success of making multi-component crystalline phases (crystalline molecular complexes or co-crystals). Such multi-component crystalline phases have proved important in the pharmaceutical area, for example, where molecular complexes are known to form that contain an active pharmaceutical ingredient and that crystallize to give a unique crystal structure containing the molecular complex. Most known co-crystals contain two components, but three component co-crystals are known. Co-crystals include systems in which one or more of the formal components is itself made of more than one identifiable molecular form. These include co-crystal components that are salts, hydrates, solvates, and the like. As for stoichiometry, the most common types of co-crystals are 1:1 and 2:1 complexes, where the ratios indicate the stoichiometric presence of co-crystal components in the molecular complex or co-crystal.

The most generally applied techniques to prepare co-crystals are crystallization by solvent evaporation and by cooling solutions containing the individual components. Commonly used solution methods for co-crystal formation use organic solvents and solution concentrations of co-crystal components (reactants) on the same molar basis as the co-crystalline product. With these methods, there is a risk of crystallizing the single component phases thereby eliminating the possibility of accessing the multi-component crystalline phase. Moreover, crystallizing by the known solution methods entails the evaluation of a number of crystallizing conditions such as choice of solvent, component concentration, rate and extent of evaporation or cooling, and the like.

### SUMMARY OF THE INVENTION

A method for preparing multi-component crystals (co-crystals) by reaction co-crystallization involves a chemical reaction or interaction of components of the co-crystal in a microphase or a macrophase and leads to formation of a crystalline product of multiple components, without the need for grinding, solvent evaporation, or temperature variation. As used herein, co-crystals refers to single phase compositions that are made of at least two components that are identifiable as different molecular forms. It does not refer to multiple crystals, i.e. crystals in which the crystallizing form is a dimer, trimer, or higher multimer of a single component.

Conditions are achieved leading to rapid co-crystallization (i.e. formation of a solid phase containing the co-crystal by precipitation) by the choice of reactant concentration in solutions, solvent, and other factors. In one aspect, reaction co-crystallization is a term given to the process by which the apparent solubility of a multi-component complex in a solvent system is decreased upon adding molar excesses of one (but not both or all) of the components of the complex. It is believed the method works in part by reducing the solubility of the molecular complex in the solvent, increasing the likelihood that the molecular complex is the least soluble form in the system, upon which it precipitates.

In various aspects, the concept of a co-crystal solubility product is advanced to explain the phase solubility diagram of the co-crystal system and identify conditions under which co-crystals can be prepared in micro- and macrophases, or alternatively identify conditions under which formation or precipitation of co-crystals is desirably avoided.

In various embodiments, methods of producing co-crystals by precipitation from a liquid phase involve combining two or more reactants (co-crystal components) together with solvent in a molar ratio such that the molar concentration of one of the reactants in solution is significantly higher than the concentration of the other reactant or reactants. In preferred embodiments, the molar excess of one reactant over the other is greater than 2:1, and is preferably at least 5:1. As noted, it is believed that the molar excess of one of the reactants reduces the solubility of the complex by a mechanism analogous to the common ion effect.

Further areas of applicability of the present invention will become apparent from the detailed description provided hereinafter. It should be understood that the detailed description and specific examples, while indicating various embodiments of the invention, are intended for purposes of illustration only and are not intended to limit the scope of the invention.

### BRIEF DESCRIPTION OF THE DRAWINGS

FIG. 1 is a phase diagram for a two component reactive system.

FIG. 2 is an illustrative plot of transition ligand concentration against solubility of component A.

FIGS. 3a and 3b are phase diagrams of systems containing a molecular complex.

FIG. 4 shows the solubility of a carbamazepine:nicotinamide co-crystal.

FIGS. 5 and 7 shows Raman spectra of component and co-crystal.

FIGS. 6 and 8 show Raman peak shift over time during formation of co-crystal.

### DETAILED DESCRIPTION OF PREFERRED EMBODIMENTS OF THE INVENTION

It is known that under various conditions some organic compounds form nonionic complexes in solution with other organic compounds. The complexes are held together with nonionic interactions such as hydrogen bonding and the like. Under certain crystallization conditions, including those in which the complex thus formed is less soluble than the other forms in the system, the molecular complex crystallizes or precipitates out of a solution to form what is referred to as a co-crystal. As used here unless the context requires otherwise, the term co-crystal refers to the precipitated solid, while the more general term molecular complex is used for the multi-component molecular complex (usually a binary or ternary complex, with binary complexes being the most common) whether in the solution or solid phase. The molecular entities that interact with one another to form the molecular complexes and the co-crystals are referred to as co-crystal components, reactants, prospective reactants, or similar terms. Although individual reactants can be provided in salt form, it is understood that the molecular complexes that are the product of the reaction co-crystallization are formed by nonionic interactions with other reactants, and do not rely on ionic interactions such as salt formation to form the complexes.

Solvents include single liquids and solvent systems containing two or more individual liquids where the liquids are aqueous or organic and act as solvent for the reactants or complexes. When separate solutions of individual reactants or components are provided, it is understood that the individual components can be in the same or in different solvent systems with respect to each other. It is further understood that while a solution of a particular reactant predominantly contains the reactant mentioned, it may also further comprise other components or minor amounts of other reactants in the system.

In some embodiments, reactants or components are provided in the physical form of slurries or suspensions. These are to be understood as containing a solid phase in contact with a liquid phase; when the component is soluble in the liquid (i.e., when the liquid acts as a solvent for the component), the liquid phase generally contains at least some dissolved solids corresponding to the reactant being used. In some aspects of the invention, the liquid phase of the slurry or suspension is saturated with respect to the reactant species present as the solid in the suspension or dispersion.

Although several aspects of the invention are described herein in theoretical chemistry terms, it is to be understood that the invention is not to be limited to the theory put forth. Theoretical considerations are presented in order to more fully describe the invention and its various uses.

The identity of the reactants or components making up the molecular complexes and co-crystals described herein is not particularly limited. However, the structure of the individual components must allow for some kind of nonionic interaction such as hydrogen bonding, dipole-dipole interactions, and the like to stabilize the complex. In various preferred embodiments of the invention, at least one of the reactants is an active pharmaceutical ingredient such as a drug or other pharmaceutical active agent. A number of molecular complexes and co-crystals of active pharmaceutical ingredients are known. In various embodiments, the invention provides methods of making known co-crystals and methods for screening to find new co-crystal systems.

In one embodiment, the invention provides a method of decreasing the solubility of a molecular complex in a solvent system. The molecular complex is made of two or more organic reactants held in complex by nonionic interactions between the reactants. In one aspect the method involves adding a stoichiometric excess of one of the reactants to a solution of the complex, or to a solution of the components in solution in a stoichiometric amount equal to the presence of the components in the co-crystal. In various embodiments, the complex is formed by conventional methods or by methods described herein. In various embodiments, the method involves adding a composition including a solid reactant to a solution of the other reactant(s) or a solution of the complex. The solid reactant can alternatively be added as a solid or as the solid phase of a suspension or slurry. In various embodiments, it is possible but not required that the multi-component complex forms in solution before it precipitates as the solid co-crystal.

The stoichiometric excess is preferably at least 2:1, more preferably 5:1, and more preferably 10:1, based on the stoichiometric presence of the reactant in the complex. For example, in the common situation where the complex is a binary (1:1) complex, it is preferred to add at least 2 moles of reactant per mole of complex, preferably at least 5, and more preferably at least 10 to achieve the respective ratios. To illustrate for a ternary (e.g. 2:1) complex, a 2:1 stoichiometric excess is achieved by providing 4 moles of the first reactant (the one present twice in the complex) to one mole of the other reactant.

Somewhat more generally, a method for precipitating a molecular complex from a solvent system, the complex comprising two or more reactants held in complex by nonionic interactions, involves adding a stoichiometric excess such as described above of one of the reactants to a solution comprising the two or more reactants. In a preferred embodiment, the solution comprising the two or more reactants contains the respective reactants at stoichiometric amounts corresponding to their presence in the complex. Preferably, the solution is relatively concentrated in at least one of the reactants. For example, at least one of the reactants is saturated.

In another embodiment, a method of producing a solid co-crystal composition by precipitation from a liquid phase is provided. As before, the molecular complex is made of two or more organic reactants stabilized by nonionic interactions between the reactants. The method involves combining the reactants and a liquid solvent under over saturation conditions with respect to the molecular complex in the solvent. One of the reactants is present in the combination in at least a 2:1 molar excess relative to its presence in the co-crystal, preferably 5:1, and more preferably 10:1 molar excess. Preferably, the reactants and liquid solvent are combined under over saturation conditions. Here and elsewhere, over saturation conditions means that the concentrations of the individual reactants in the reactant/solvent system are such that when a molecular complex forms, it is formed at a concentration at or above its solubility in the solvent. As noted herein, it is believed that providing one of the reactants (not both or all) in a molar excess, preferably a significant molar excess, leads to lower solubility of the molecular complex in the solvent, and an increased likelihood that over saturation conditions are reached with respect to the complex. Accordingly, it is preferred to provide at least one of the reactants and preferably both in relatively concentrated forms.

Here and in other embodiments, when the solubilities of the reactants in the solvent used differ, highly concentrated or even saturated solutions of both of the reactants can be used. This provides not only a high concentration to achieve over saturation conditions, but also a high molar excess of one of the components, which is believed to lower the solubility of the complex in the solvent and lead to its precipitation as a co-crystal.

In another embodiment, the method of the invention provides for making pharmaceutical co-crystals by precipitation of a solid form from a solvent. The pharmaceutical co-crystal is a solid molecular complex between two or more reactants held together by nonionic interaction, wherein one of the reactants is an active pharmaceutical ingredient. The method involves combining the active pharmaceutical ingredient, other reactants and solvent under over saturation or supersaturation conditions with respect to the complex in the solution. As before, at least one of the active pharmaceutical ingredients and the other reactant or reactants is provided in a molar excess of at least 2:1 with respect to its presence in the complex, preferably at least 5:1, and more preferably at least 10:1. With respect to identity of the individual reactants, the component provided in molar excess in various embodiments is either the active pharmaceutical ingredient or another reactant.

In another embodiment, the reaction co-crystallization methods of the invention are used to screen for the formation of a nonionic molecular complex from prospective reactants. In various embodiments, the method is used to identify conditions under which known or prospective complexes precipitate (or do not precipitate) from various solvents. Alternatively or in addition, the screening methods are used to discover or synthesize new complexes or co-crystals. Thus, pairs, trios, or other combinations of prospective reactants are tested in the method. Prospective reactants are combined in a solvent, wherein one of the prospective reactants is present in solution at a molar excess, preferably at a ratio of at least 2:1 with respect to another reactant, based on the molar or stoichiometric presence of the reactant in the co-crystal. Upon combining the prospective reactants, the system is observed. If a precipitate forms, the precipitate is analyzed to determine or confirm that a co-crystal form comprising the reactants has come out of solution. In various embodiments, the precipitate is analyzed by Raman spectroscopy, infrared spectroscopy, x-ray diffraction, or other suitable procedures. In some embodiments, a co-crystal phase (precipitated molecular complex) exhibits a different Raman absorption spectrum than either of the reactants. In a non-limiting example, the presence of a reactant in a co-crystalline form is detected by observing shifts in Raman peaks or infrared absorption bands that can be on the order of 1 to 10 wave numbers. In the case of x-ray diffraction, it is well known that co-crystals crystallize in different unit cells than the reactants from which the complexes are formed. The different unit cell dimensions can be determined by x-ray diffraction methods such as powder diffraction.

Here and in other embodiments, when the individual prospective reactants have different solubilities in the solvent or solvent system to be used, the reactants are preferably provided at relatively concentrated levels, up to and including saturation in the solvent. Where the solubilities differ widely, saturated solutions of the individual reactants provide suitable molar ratios of at least 2:1, preferably at least 5:1, and more preferably at least 10:1 of the prospective reactants in the solvent (all ratios are in relation to the molar presence of the respective reactants in the molecular complex). As noted, the use of relatively concentrated solutions of the individual reactants increases the likelihood that the molecular complex formed in solution is at or above its solubility limit, or that over saturation conditions are achieved. Also as noted, the use of prospective reactants where one is in a molar excess lowers the solubility of the complex and increases the likelihood that the complex is the least soluble form in the solution, leading to its precipitation.

In various embodiments, the methods of the invention involve combining reactants in a solvent system in such a way that the stoichiometric presence of one of the reactants in solution is greater than the other reactant or reactants, when measured relative to the stoichiometric presence of the reactant component in the complex to be formed. This imbalance of stoichiometry in solution is believed to lead to precipitation of molecular complexes (co-crystals), as discussed theoretically below. The reactants are combined in various forms: as solutions, as slurries, or as solids. In various embodiments, a liquid or a vapor is brought into contact with solid ingredients. Differential solubility of the reactants in the liquid solvent (or the vapor as is adsorbs on to the solid) leads to non-stoichiometric concentrations of reactants in solution, which lead to enhanced precipitation and isolation of co-crystal.

In various embodiments, combining the reactants with solvent is accomplished by sorption. Sorption is the spontaneous acquisition of a component (water, ethanol or another solvent) from the atmosphere or vapor phase. Sorption of a component from the vapor can take the form of a condensed phase and can serve as a solvent. For instance, in the case of water, many water soluble substances have been shown to spontaneously dissolve when exposed to relative humidity above a critical value, where adsorbed water serves as a solvent. The sorbed phase provides a microphase for reaction co-crystallization as a result of dissolution of reactants, non-stoichiometric concentrations of reactants in solution and precipitation of the molecular complex in crystal form. The reaction mediated by vapor sorption onto a solid (solvents such as ethanol, water, and others) or by a deliquescent behavior will proceed to completion, or consume the reactants if the reactants in solid state are in the same stoichiometry as in the molecular complex to be crystallized. For crystallization of the molecular complex, non-stoichiometric solutions (i.e. those in which there is a molar excess of one of the reactants in solution) are achieved by different dissolution rates or solubilities of the reactants in the sorbed phase or solvent film.

In preferred embodiments, the co-crystalline product of the invention contains pharmaceutical components or active pharmaceutical ingredients. Advantageously, the invention provides a process where the reaction co-crystallization proceeds with various solid state forms of the reactants, such as polymorphs, salts, hydrates, solvates, amorphous, or crystalline solid state forms.

In various embodiments, the solid state forms of the reactants are used in the solid state, in slurries in contact with a liquid phase, or in solution. It is to be understood that stoichiometric excess refers to the solution concentration of reactants. Therefore reactants, when all reactants are in solid forms, can have the stoichiometric composition in solid phase equal to that of the co-crystal; in such a case, however, non-stoichiometric solution concentrations are achieved by different dissolution rates of each reactant in the solvent.

In various embodiments, the invention provides batch and continuous co-crystallization reactions by, for example, slurrying one or more of the reactants in solvent or solutions of reactant(s) to a suspension (slurry) of reactant(s), and for adding pure solvent to solid reactant, including contacting solvent with solid reactants or adding solvent in a larger phase to prepare a slurry of the reactants.

In various embodiments, methods of the invention are carried out by combining two or more reactants with a solvent to form a reaction co-crystallization system.

In various embodiments, reaction co-crystallization is carried out by combining various streams into suitable reactors, vessels, mixers, and the like. The various streams together comprise the reactants (co-crystalline components) and solvent. Normally, individual streams before combination comprise predominantly one or another reactant. For example (illustrating with a complex containing two components A and B), a first stream comprising co-crystal component A is provided, and a second stream comprising co-crystal component B is provided. For co-crystals containing more than two components a third stream containing co-crystal components C is provided. In preferred embodiments, the respective streams before combination contain only the respective co-crystal component; alternatively, the individual streams contain other co-crystal components (reactants). While the method is general for ternary and higher complexes, the method is further described herein for illustrative purposes with reference to a binary system containing two reactants A and B.

Streams comprising reactants A or B are provided as a pure solid, as a solution of the respective reactant in a solvent, or as a suspension or slurry. When provided as a suspension, the streams contain solid reactants in contact with a solvent phase, the solvent phase normally containing dissolved reactant. For example, a slurry stream contains a reactant in contact with a saturated solution of the reactant in the solvent. When the streams collectively containing reactants A and B are combined, a reaction co-crystallization mixture is produced that contains reactant A, reactant B, and a solvent.

Any manner of combining the streams can be used, as long as a reaction co-crystallization mixture containing the reactions and solvents is produced. The co-crystallization mixture is a solution of reactants in which the presence of one of the reactants is in a molar excess relative to its presence in the complex formed from nonionic interactions between the reactants. As noted, the molar excess is preferably 2:1 or greater in solution. For example, in some embodiments, solutions of reactants, which in various embodiments are premixed prior to combination, are fed into a reactor or vessel. In various embodiments, the solutions are saturated, supersaturated, or undersaturated at the temperature of feeding. If desired, the solutions are prepared warm and allowed to cool prior to or after addition to the vessel. In this way, over saturation or supersaturation conditions are readily achieved. In a batch process, the reaction co-crystallization mixture is formed in a reactor or vessel and a precipitate containing the co-crystalline composition is isolated by draining or filtering.

In various embodiments, continuous processes are carried out, for example in a tubular reactor or vessel. Streams collectively containing reactants A and B are continuously pumped into a reactor where reaction takes place and co-crystals are precipitated. The precipitate containing the co-crystalline composition is continuously renewed by filtration or other means.

Screening methods take advantage of the increased efficiency of precipitation and higher yields brought about by combining co-crystal component non-stoichiometrically as described herein. In one aspect, screening involves subjecting a complex or a co-crystal system to a series of conditions to determine whether and under what conditions a co-crystal precipitate is formed and isolated. Conditions to be evaluated include, without limitation, nature of the co-reactant, solvent, yield of co-crystal precipitate, molar ratio of the reactants, and so on. The methods are adaptable to high throughput operations and/or robotic automation as desired. In one embodiment, the screening methods are carried out in conventional equipment, such as an industry standard 96-well plastic tray.

In various embodiments, screening methods involve variation of co-crystallization conditions according to a predetermined or pre-set plan for probing the response of the co-crystallization system to experimental variables. In a non-limiting embodiment, the plan is to select an active pharmaceutical agent and probe what organic molecules form an isolatable co-crystal upon precipitation from a solvent system. Here a series of reaction vessels (which can be the individual wells of a multi-well plate) are provided with a solution, slurry, or solid comprising the active pharmaceutical agent of interest. Then, a series of test solutions, test slurries, or test solids is combined with the pharmaceutical agents in the respective reaction vessels. The experimental conditions are selected such that, upon combination of the prospective reactants, one of the reactants is in a molar excess, preferably of at 2:1, and more preferably at least 5:1, with respect to its molar presence in the complex. The use of a stoichiometric excess of one of the reactants increases the likelihood that any nonionic complex formed will be the least soluble form in the system, and so will precipitate out as a co-crystal.

The nature of any precipitate is then probed by any of a number of suitable analytic techniques, such as without limitation Raman spectroscopy, infrared spectroscopy, FTIR spectroscopy, and x-ray diffraction. In various embodiments, the analysis involves determination of the structure of the precipitate (e.g. to demonstrate that the precipitate is a co-crystalline form and not just one or other of the reactants). Especially when the co-crystal or co-crystal system under investigation is known, the analysis can be limited to a confirmation that the co-crystal precipitate at hand is the same as that noted before. For example, analysis can be limited to a region of the spectrum of diffraction pattern known to contain diagnostic peaks, such as a particular Raman or infrared band, or a particular set of diffraction peaks indicative of the structure. In some embodiments, the precipitate is analyzed at a single wave length or diffraction angle to determine, at least on a first pass, whether the precipitate is of interest or whether the particular conditions are worthy of further study.

The methods also lend themselves to use in combinatorial screening. For simplicity, the method is described for a binary (two-component) co-crystal system, but the results are readily generalized to three-component and higher systems. Instead of a single component A being combined with a single component B, and any resulting precipitate being analyzed to confirm or determine whether the A:B is formed, the streams containing A can instead contain a plurality of components A₁, A₂, A₃ . . . Aᵢ. Similarly, the streams containing B optionally contain a plurality of components B₁, B₂, B₃ . . . Bⱼ. In some embodiments, a plurality of components Aᵢ is screened with a single component B, and vice versa. For example, component A comprises a drug or drugs of interest, and component B comprises a potential complex former or collection of potential complex formers with the active drug(s) A. In some embodiments, a plurality of components Aᵢ is screened with a plurality of components Bⱼ.

The compositions of the respective combinatorial libraries of potential reactants, the solubility of individual components in the solvents chosen, the nature of the complexes formed (e.g. whether binary, ternary, or higher), and other factors determine the conditions under which the screening is to take place. The key is to provide the potential reactants in a stoichiometrically unbalanced way (i.e. with one of the complex formers or potential complex formers in stoichiometric excess, preferably 2:1 or greater, relative to its presence in the co-crystal), in order to increase the likelihood that a complex formed will have lower solubility than the reactants, and thus preferentially precipitate.

When the screening is carried out combinatorially and a positive result is achieved (i.e. when a precipitate is observed) in a particular reaction vessel, generally the system needs to be studied further to determine which of the reactants Aᵢ and Bⱼ (and Cₖ when ternary systems are investigated) were responsible for the precipitate formation. As appropriate, further combinatorial or one-on-one screening is carried out in a further investigation.

In various embodiments, combinatorial screenings are carried out to determine conditions under which a complex formation is desirably avoided. To illustrate, it is sometimes useful to determine whether a particular active ingredient is capable of forming a new solid form in combination with any of the ingredients the active comes into contact with during synthesis, compounding, or administration. To probe this, a single reactant A (for example, the active ingredient of interest) is combinatorially screened with a number of components Bⱼ.

Co-crystals containing an active pharmaceutical ingredient (API) as one component or reactant are known and include the co-crystals listed in the table. The “co-reactant” along with the API is referred to as a “ligand” in the table. The table also indicates the composition of the complex as a ratio of API:ligand. This ratio gives the stoichiometric presence of the individual reactants in the complex.

| | | Ratio |
|---|---|---|
| API | Ligand | (API:Ligand) |
| Carbamazepine | Nicotinamide | 1:1 |
| | Saccharin | 1:1 |
| | Benzoquinone | 1:1 |
| | Terephthalaldehyde | 1:1 |
| | Acetic acid | 1:1 |
| | Formic acid | 1:1 |
| | Butyric acid | 1:1 |
| | Trimesic acid | 1:1 |
| | 5-nitroisophthalic acid | 1:1 |
| | Adamantane-1,3,5,7- | 1:1 |
| | tetracarboxylic acid | |
| | Formamide | 1:1 |
| Caffeine | Malonic acid | 2:1 |
| | Oxalic acid | 2:1 |
| | Glutaric acid | 1:1 |
| | Maleic acid | 2:1, 1:1 |
| | Benzoic acid | 1:1 |
| | Salicylic acid | 1:1 |
| | p-hydroxybenzoic acid | 1:1 |
| | m-hydroxybenzoic acid | 1:1 |
| | Gentisic acid | 1:1, 1:2 |
| Itraconazole | Succinic acid | 2:1 |
| | Malic acid | 2:1 |
| | Tartaric acid | 2:1 |
| | Fumaric acid | 2:1 |
| Fluoxetine | Succinic acid | 2:1 |
| hydrochloride | Fumaric acid | 2:1 |
| | Benzoic acid | 1:1 |
| Aspirin | 4,4′-bipyridine | 2:1 |
| Ibuprofen | 4,4′-bipyridine | 2:1 |
| Flurbiprofen | 4,4′-bipyridine | 2:1 |
| | trans-1,2-bis(4- | 2:1 |
| | pyridyl)ethylene | |
| | 4,4′-dipyridylethane | 2:1 |
| Sulfamethazine | Benzoic acid | 1:1 |
| | Salicylic acid | 1:1 |
| | Anthranilic acid | 1:1 |
| | Acetylsalicylic acid | 1:1 |
| | o-phthalic acid | 1:1 |
| | p-chlorobenzoic acid | 1:1 |
| | p-aminobenzoic acid | 1:1 |
| | p-aminosalicylic acid | 1:1 |
| | Aminacrine | 1:1 |
| | Trimethoprim (methanolate) | 1:1 |
| | Trimethoprim (monohydrate) | 1:2 |
| Sulfamethoxypyridazine | Trimethoprim | 1:1 |
| Sulfametrole | Tetroxoprim (hydrate, | 1:1 |
| | ethanolate, methanolate) | |
| | Trimethoprim | 1:1 |
| Sulfamethoxazole | Trimethoprim | 1:1 |
| Theophylline | Ethylenediamine | 1:1, 2:1 |
| | 1,10-phenanthroline | 1:1 |
| | ethylenediamine carbamate | 1:1 |
| | Salicylic acid | 1:1 |
| | 5-sulfosalicylic acid | 1:1 |
| | (hydrate) | |
| | p-nitroaniline | 1:1 |
| | Urea | 1:1 |
| | Sulfathiazole | 1:1 |
| | 5-chlorosalicylic acid | 1:1 |
| Mebandazole | Propionic acid | 1:1 |

The case of fluoxetine hydrochloride illustrates a case where one of the reactants that make up a molecular complex is itself a kind of multi-component system, here a salt. The complex is held together by nonionic interactions between the respective ligands and the salt. It is also noted that several of the ligands in the table above are carboxylic acids. The complexes are formed under conditions of solvent and pH where a salt does not form upon complexation. For example, salts of carboxylic acids and weak amine bases generally do not form in non-aqueous solvents. In aqueous solutions, the pH determines the ionization state of weak acids and weak bases, according to known principles. In the table, the carboxylic acid ligands illustrate suitable functional groups that can form nonionic interactions by hydrogen bonding, dipole-dipole interactions, and the like.

Although the invention is not to be limited by theory, the solubility of a binary co-crystal of API (A) and ligand or co-crystal component (B), of composition Aₐ:B_{b} where the co-crystal components do not ionize or form complexes in solution, is given by the equilibrium reaction 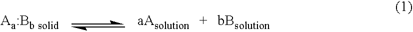
Subscripts refer to the stoichiometric number of molecules of A or B in the complex. The equilibrium constant for this reaction is given by

$${K}_{eq} = \frac{{a}_{A}^{a} {a}_{B}^{b}}{{a}_{A \text{:} B}} \qquad ( 2 )$$

and is proportional to the thermodynamic activity product of the co-crystal components. If the activity of the solid is equal to 1 or is constant, the co-crystal solubility can be described by a solubility product
Kₛₚ=a_{A}^{a}a_{B}^{b}[A]^{a}[B]^{b} (3)
where [A] and [B] are the molar concentrations of each co-crystal component at equilibrium, as long as the activity coefficients are unity. This approximation applies to dilute solutions and for practical purposes will be used in this manuscript to calculate material balances and solution compositions.

If a binary co-crystal of 1:1 stoichiometry dissolves in pure solvent into its individual components without further complexation or ionization to form a saturated solution, the mass balance for each component in solution can be expressed in terms of the molar solubility of the co-crystal, S
[A]=S and [B]=S, (4)
and substituting these in the solubility product equation (3) gives
Kₛₚ=S² and S=(Kₛₚ)^{1/2} (5)
Equations 4 and 5 apply only to solutions of stoichiometric composition when the solution molar ratio is the same as that of the co-crystal.

For non-stoichiometric solution compositions, let C be the excess concentration of ligand so the mass balances when excess B is added become
[A]=S and [B]=S+C, (6)
and therefore,
Kₛₚ=S(S+C) (7)
In the case where a large excess of ligand is present, such that C>>S, then
S≈Kₛₚ/C. (8)
A quadratic equation must otherwise be solved, and gives

$$S = \frac{- C + \sqrt{{C}^{2} + 4 {K}_{sp}}}{2} \qquad ( 9 )$$

This equation predicts that addition of either co-crystal component to a solution in excess of S decreases the co-crystal solubility when the preceding conditions apply.

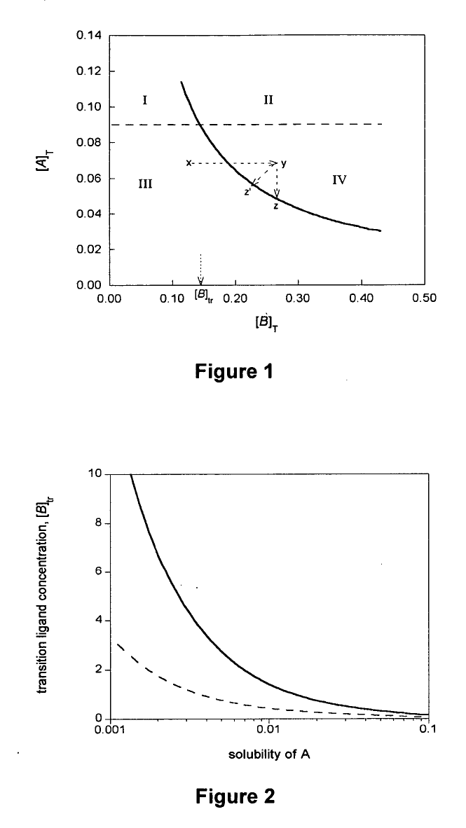

A plot of the solubility of co-crystal A:B as a function of total ligand in solution according to equation (9) is shown in FIG. 1.

FIG. 1 shows the effect of ligand concentration on the solubility of co-crystal A:B (solid line) and the solubility of single component crystal A (dashed line) calculated from equation (9) with Kₛₚ=0.0129 M², and S_{A}=0.09 M. The transition ligand concentration [B]ₜᵣ, occurs when the solubility of A equals the solubility of A:B.

Here, [A]_{T}=S and [B]_{T}=S+C as shown in equation (6), and the subscript “T” stands for total concentration. This solubility behavior resembles that of the common ion effect in the case of sparingly soluble salts. But in contrast with the case of salts and that of solvates where analogous equilibria have been considered, co-crystals dissociate into primary components (at least two different molecules) that can crystallize as single component phases.

Also shown in FIG. 1 is the solubility of single component crystal of A, as a function of co-crystal component or ligand (B) concentration in solution. This phase diagram is based on the following assumptions: (1) A is less soluble than B, (2) A is less soluble than A:B in stoichiometric solutions (with respect to A:B), (3) there is no complexation or ionization of co-crystal components in solution, and (4) the solubility of A is independent of the concentration of B in solution. Under these considerations, the solubility curves of co-crystal and single component crystal intersect. Therefore, there is a co-crystal component concentration in solution, [B]ₜᵣ, at which the solubility of co-crystal A:B is equal to the solubility of crystal A and above which the solubility of co-crystal A:B is less soluble than crystal A.

The transition concentration of co-crystal component can be predicted by substituting the single component crystal solubility, S_{A}, for the co-crystal solubility, S, in equation (7) and rearranging to give

$${C}_{tr} = \frac{{K}_{sp} - {S}_{A}^{2}}{{S}_{A}} \qquad ( 10 )$$

where Cₜᵣ is the excess concentration of co-crystal component at the transition concentration, and the total concentration of co-crystal component at the transition, [B]ₜᵣ, is given by

$${[ B ]}_{tr} = S + {C}_{tr} = S + \frac{{K}_{sp} - {S}_{A}^{2}}{{S}_{A}} \qquad ( 11 )$$

where S is the solubility of co-crystal under stoichiometric conditions. This equation predicts that [B]ₜᵣ increases as the solubility of A decreases or as Kₛₚ increases, as shown in FIG. 2. FIG. 2 shows the transition ligand concentration as a function of the solubility of the single component crystal, S_{A}, calculated from equation (11) with values of Kₛₚ=0.0129 M² (solid line) and Kₚ=0.0032 M² (dashed line).

The phase diagram which includes the solubilities of co-crystal and single component crystal, FIG. 1, also defines four domains representing regions of kinetic and thermodynamic control for the dissolution or crystallization of the single and multi-component phases. Domain I is supersaturated with respect to A but undersaturated with respect to co-crystal A:B. Both A and A:B are supersaturated in domain II, but undersaturated in domain III. Domain IV is supersaturated with respect to A:B but is undersaturated with respect to A. A theoretical plot such as this indicates regions of thermodynamic stability and which form(s) will dissolve or have the potential to crystallize. This information is important for the development of screening methods for the crystallization of co-crystals, identifying conditions where phase transformations between crystal and co-crystal occur, and controlling or preventing the crystallization of co-crystal in solutions of co-crystal components.

FIG. 1 also illustrates the path of a solution that is initially undersaturated with respect to A and A:B at point x and goes through saturated ([A]_{T}=S_{A:B}) and supersaturated ([A]_{T}>S_{A:B}) states with respect to A:B as the ligand concentration in solution increases to point y. The driving force for crystallization is the supersaturation, or difference in chemical potential between y and z. Since crystallization of A:B will reduce [B], the reaction will proceed until a saturated state at z′ is reached.

While the decrease in co-crystal solubility with increasing ligand concentration provides a means for identifying conditions for preparing co-crystals, the path shown in FIG. 1, x to y to z′, predicts the unexpected crystallization of A:B from a formulation of compound A in solutions of B if only the solubility of A is considered during development. For instance, in the case where an API (A) is formulated in an undersaturated solution of composition x addition of excipient B to the solution at a composition y may result in crystallization of co-crystal A:B, since the concentration y in domain IV is supersaturated with respect to the co-crystal.

Effect of Complex Formation in Solution on the Solubility of Co-Crystals A:B (1:1)

1:1 Solution Complexation

When dissolution of a 1:1 co-crystal of an API (A) and ligand (B) leads to 1:1 complex formation in solution, the equilibrium reactions are 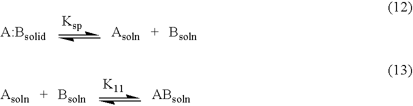
Equilibrium constants for these reactions are the solubility product
Kₛₚ=[A][B] (14)
and the binding constant for the 1:1 complex formed in solution

$${K}_{11} = \frac{[ AB ]}{[ A ] [ B ]} = \frac{[ AB ]}{{K}_{sp}} \qquad ( 15 )$$

From the mass balances for A and B in solution
[A]_{T}=[A]+[AB] (16)
[B]_{T}=[B]+[AB] (17)
[AB] is the solution concentration of complex and according to Equation (15) is
[AB]=K₁₁Kₛₚ (18)
Thus the solution concentration of complex is fixed by the coupled equilibria. This presents an unusual and interesting condition.

Substituting equations (14) and (18) into equations (16) and (17) one obtains

$$\begin{matrix}{[ A ]}_{T} = \frac{{K}_{sp}}{[ B ]} + {K}_{11} {K}_{sp} & ( 19 ) \\ {[ B ]}_{T} = [ B ] + {K}_{11} {K}_{sp} & ( 20 )\end{matrix}$$

[A]_{T} is the solubility of co-crystal A.B, when measuring total A in solutions under the equilibrium conditions described in equation (1). By combining the above equations, the co-crystal solubility can be expressed in terms of the total ligand concentration, [B]_{T} according to

$$\begin{matrix}{[ A ]}_{T} = \frac{{K}_{sp}}{{[ B ]}_{T} - {K}_{11} {K}_{sp}} + {K}_{11 } {K}_{sp} \\ If {K}_{11} {K}_{sp} << {[ B ]}_{T} , then & ( 21 ) \\ {[ A ]}_{T} = \frac{{K}_{sp}}{{[ B ]}_{T}} + {K}_{11} {K}_{sp} & ( 22 )\end{matrix}$$

Therefore, both Kₛₚ and K₁₁ can be evaluated from a plot of [A]_{T} versus 1/[B]_{T}. If there are no higher order complexes in solution, this plot is linear with
slope=Kₛₚ
and
intercept=K₁₁Kₛₚ
under the conditions K₁₁_{K}ₛₚ<<[B]_{T}.

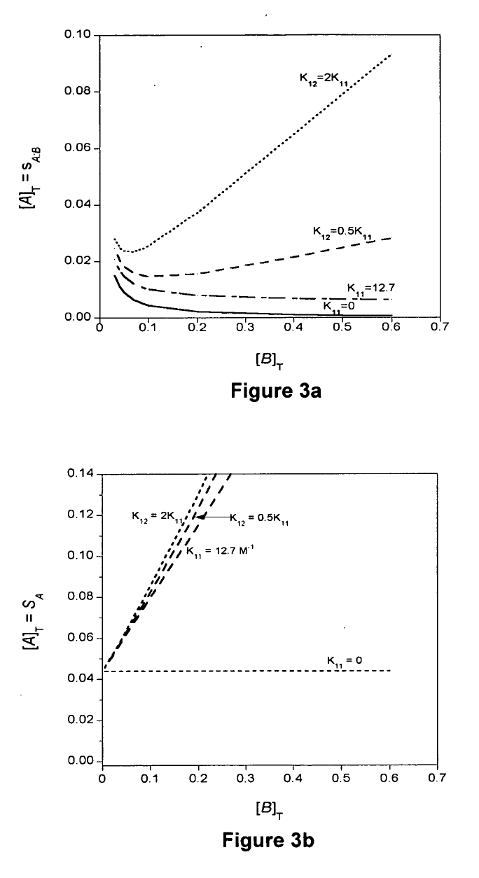

Equation (22) predicts that co-crystal solubility decreases with increasing ligand concentrations and that co-crystal solubility is higher by a constant value, the product of K₁₁ and Kₛₚ compared to the case where there is no solution complexation. This is shown in the lower two curves of FIG. 3a. The top two curves represent conditions with higher order complexes, 1:2, and are discussed in the following section.

FIG. 3 shows the effect of solution complexation on the solubility of (FIG. 3a) co-crystal A:B and (FIG. 3b) single component crystal A as a function of total ligand concentration. The co-crystal solubility was calculated from equation (22) or (32) depending on the solution complex stoichiometry as discussed in the text: no complex, 1:1 complex, and 1:1+1:2 complexes. Co-crystal Kₛₚ=4.5×10⁻⁴ M² and the complexation constant values used are indicated in the graph. A minimum co-crystal solubility is predicted when a 1:2 complex is formed and this concentration can be calculated from equation (35). Single component crystal solubility was calculated from equation (25) or (36) considering the same solution complex stoichiometries.

The K₁₁ determined from the binary co-crystal solubility as a function of ligand concentration can also be used to predict the solubility increase of single component crystal (A) in solutions of ligand, according to 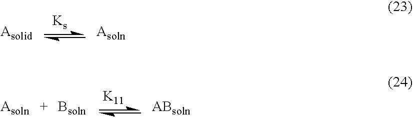
where Kₛ is the equilibrium constant for the solubility of crystal A, and K₁₁ is the binding constant for the formation of the complex AB in solution. The equilibrium reaction for complex formation in solution, equation (24), is the same as that in the case of binary co-crystals presented above, equation (13). Using the mass balance for total A and total B, the solubility of the single component A is given by

$${[ A ]}_{T} = {[ A ]}_{0} + \frac{{{{K}_{11} [ A ]}_{0} [ B ]}_{T}}{1 + {{K}_{11} [ A ]}_{0}} \qquad ( 25 )$$

where [A]₀ is the intrinsic solubility of crystal A in the absence of ligand. When the solubility of co-crystal in solutions of ligand is lower than that of the single component crystal, direct measurement of the solubility of A may not be possible due to crystallization of co-crystal. Therefore, this method provides a means of predicting the solubility of A in the presence of ligand.
1:1 and 1:2 Solution Complexation

The dependence of co-crystal A:B solubility on ligand concentration will reveal the presence of 1:1 and 1:2 solution complexes as described below. Consider a 1:2 complex of A and B formed by bimolecular collisions where B binds to AB to form the soluble complex AB₂. The equilibrium expression for the formation of the AB₂ complex is 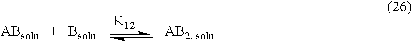
The equilibria described by equations (12) and (13) lead to formation of the 1:2 complex in a stepwise fashion, and by taking into account the equilibrium constants for these reactions, K₁₂ can be expressed in terms of K₁₁ and Kₛₚ by

$${K}_{12} = \frac{[ {AB}_{2} ]}{[ AB ] [ B ]} = \frac{[ {AB}_{2} ]}{{K}_{11} {K}_{sp} [ B ]} \qquad ( 27 )$$

The mass balance of A now becomes
[A]_{T}=[A]+[AB]+[AB₂] (28)
Substituting expressions developed for Kₛₚ, K₁₁, and K₁₂ into this equation leads to an expression for [A]_{T} in terms of the free ligand concentrations and equilibrium constants by

$${[ A ]}_{T} = \frac{{K}_{sp}}{[ B ]} + {K}_{11} {K}_{sp} + {K}_{11} {K}_{12} {K}_{sp} [ B ] \qquad ( 29 )$$

If [B] is not known, the expression for total ligand concentration in solution
[B]_{T}=[B]+K₁₁Kₛₚ+2K₁₁K₁₂Kₛₚ[B] (30)
can be rearranged and substituted into the mass balance equation for [A]_{T}, equation (29) obtaining

$${[ A ]}_{T} = \frac{{K}_{sp} ( 1 + 2 {K}_{11} {K}_{12} {K}_{sp} )}{{[ B ]}_{T} - {K}_{11} {K}_{sp}} + {K}_{11} {K}_{sp} + \frac{{K}_{11} {K}_{12} {K}_{sp} ( {[ B ]}_{T} - {K}_{11} {K}_{sp} )}{1 + 2 {K}_{11} {K}_{12} {K}_{sp}} \qquad ( 31 )$$

This equation can be simplified for the case where K₁₁Kₛₚ<<[B]_{T}, and 2 K₁₂K₁₁Kₛₚ<<1 to give

$${[ A ]}_{T} = \frac{{K}_{sp}}{{[ B ]}_{T}} + {K}_{11} {K}_{sp} + {K}_{11} {K}_{12} {{K}_{sp} [ B ]}_{T} \qquad ( 32 )$$

To gain information about the shape of this curve, the first derivative d[A]_{T}/d[B]_{T} is examined and reveals that that a plot of [A]_{T} vs. [B]_{T} would be concave upward according to the change in slope from negative to positive, corresponding to low values of [B]_{T} and high values of [B]_{T}, respectively.

The total ligand concentration at which the co-crystal has the minimum solubility ([A]_{T} has the minimum value) is calculated from

$$\frac{ⅆ {[ A ]}_{T}}{ⅆ {[ B ]}_{T}} = 0 when {K}_{11} {K}_{12} {K}_{sp} = \frac{{K}_{sp}}{{[ B ]}_{T}^{2}} \qquad ( 33 )$$

and solving for [B]_{T} gives

$${[ B ]}_{T} = \sqrt{\frac{1}{{K}_{11} {K}_{12}}} \qquad ( 34 )$$

The minimum solubility of co-crystal is then obtained by substituting equation (34) in equation (32) and solving for [B]_{T} to give
[A]_{T,min}=Kₛₚ(K₁₁+2\sqrt{K₁₁K₁₂}) (35)
Thus the concentration of ligand at which the minimum co-crystal solubility occurs is inversely proportional to the binding constants and independent of the solubility product, whereas the minimum co-crystal solubility is directly proportional to the solubility product and the binding constants. This behavior is shown in the plot of [A]_{T} vs. [B]_{T} for a hypothetical system using equations (30) and (31) and assuming that Kₛₚ=4.5×10⁻⁴ M², K₁₁=12.7 M⁻¹, and K₁₂=0.5 K₁₁ or K₁₂=2 K₁₁, FIG. 3a.

The solubility of single component crystal of A is also dependent on the complexation behavior in solution. For 1:1 and 1:2 complex formation in solution and based on the equilibrium reactions (equations 24 and 26), the solubility of A is given by

$${[ A ]}_{T} = {[ A ]}_{o} + {[ B ]}_{T} ( \frac{{{K}_{11} [ A ]}_{o} + {K}_{12} {{K}_{11} [ A ]}_{o} [ B ]}{1 + {{K}_{11} [ A ]}_{o} + 2 {K}_{12} {{K}_{11} [ A ]}_{o} [ B ]} ) \qquad ( 36 )$$

This equation predicts that the solubility of A increases in a nonlinear fashion as [B]_{T} increases, since the slope is a function of [B]. A plot of [A]_{T} vs. [B]_{T} according to this equation is shown in FIG. 3b by calculating [B] from the mass balance equation and binding constants according to
[B]_{T}=[B]+K₁₁[A]₀[B]+2K₁₁K₁₂[A]₀[B]² (37)
and solving for [B] from the quadratic equation. Values for the binding constant are the same as those used for the prediction of co-crystal solubilities and are K₁₁=12.7 M⁻¹, K₁₂=0.5 K₁₁ or K₁₂=2 K₁₁.

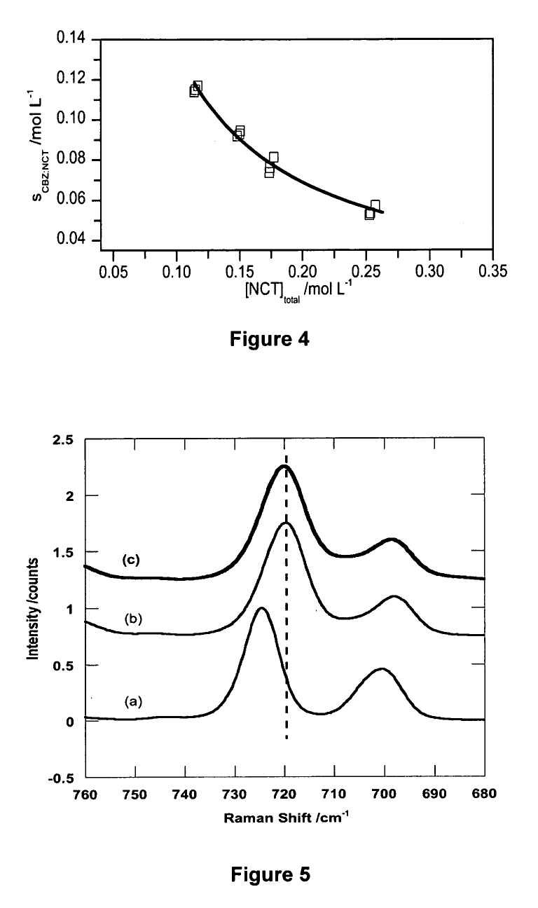

The predicted reduction in solubility of the co-crystal is borne out experimentally as illustrated, for example in FIG. 4, which uses the carbamazepine nicotinamide 1:1 co-crystal (CBZ:NCT) for illustration. The CBZ:NCT co-crystal is known; solubilities for the co-crystal and the reactants CBZ and NCT in three solvents (ethanol, 2-propylenol, and acetate) are given in the table.

| | Ethanol | 2-Propanol | Ethyl acetate |
|---|---|---|---|
| Compound | (mol L⁻¹) | (mol L⁻¹) | (mol L⁻¹) |
| CBZ:NCT | 0.116 ± 0.003 | 0.044 ± 0.003 | 0.024 ± 0.001 |
| (1:1) | | | |
| CBZ(III) | 0.1080 ± 0.0001 | 0.039 ± 0.003 | 0.0440 ± 0.0001 |
| NCT(I) | 0.841 ± 0.008 | 0.496 ± 0.004 | 0.098 ± 0.002 |

Solubilities are given at 25° C. and are expressed as the mean +/− the deviation for a sample size of 3. It is noted that only in ethyl acetate is the co-crystal the least soluble component. Accordingly, the co-crystal can be precipitated from equimolar solutions of CBZ and NCT in ethyl acetate to produce the co-crystal by cooling or evaporation methods.

In FIG. 4, co-crystal solubility is measured by adding co-crystal to pure solvent and to solutions of NCT at 25° C. The co-crystal solubility is determined by UV spectroscopy; saturation is achieved within 48 hours. Analysis of the solid phases by X-ray powder diffraction confirms there is no change in the co-crystal phase during solubility measurements.

The solubility product of CBZ:NCT co-crystal in ethanol solutions is evaluated to be Kₛₚ=0.0129 mol² L⁻² from the linear form of Equation 3, using the measured co-crystal solubilities shown in FIG. 4. The solid line of FIG. 4 represents the predicted solubility according to Equation 3 with that solubility product. In one aspect, the agreement with the experimental data demonstrates there is a common co-crystal component effect on co-crystal solubility analogous to that of the common ion effect in the case of sparingly soluble salts. But in contrast with the case of salts and that of solvates, wherein analogous equilibria can be considered, co-crystals dissociate into primary components that can crystallize into individual component phases.

### EXAMPLES

Anhydrous carbamazepine (CBZ) of USP grade and 99% purity is purchased from Sigma Chemical Company, stored at 5° C. over anhydrous calcium sulfate and used as received. Carbamazepine dihydrate is crystallized from water.

Nicotinamide (NCT) is purchased from Sigma and used as received.

Water is filtered through a double de-ionized purification system.

Ethyl acetate and 2-propanol are HPLC grade and purchased from Fisher Scientific.

Anhydrous ethanol (200 proof) is USP grade.

Anthranilic acid and saccharin are purchased from Sigma and used as received.

### Example 1

Carbamazepine:Nicotinamide co-crystal (CBZ:NCT) is obtained by mixing solutions of reactants in ethyl acetate at room temperature: 1.946 mL of an ethyl acetate solution of NCT (0.098M) is added to 4.054 mL of an ethyl acetate solution of CBZ (0.044M). After initial gentle mixing, solutions are unstirred and co-crystals are observed within 45 to 120 minutes. The Raman spectra of FIG. 5 confirms that the solid phase crystallized from solution is CBZ:NCT co-crystal. Curve a) is CBZ(III) reference; curve b) is CBZ:NCT co-crystal reference and curve c) is the co-crystal of Example 1 after drying. The peak at 720 cm⁻¹ is that of the CBZ:NCT co-crystal while the 724 cm⁻¹ peak is that of pure anhydrous CBZ(III), the monoclinic polymorph.

Co-crystal is also prepared by this method in ethanol and 2-propanol at room temperature. For example: Ethanol solution of NCT (2.25 mL of 0.8 M) is added to an ethanol solution of CBZ (3.75 mL of 0. 1 M). After initial gentle mixing, the solution was left unstirred and co-crystals were observed within 10 to 25 minutes.

Faster crystallization rates and higher yields are obtained by increasing the concentration of reactants in solution. This can be achieved by dissolving reactants in solution such that the mixed solution is saturated with respect to each reactant at the reaction temperature. At saturation, crystallization does not occur, so this method will avoid crystallization of reactants at the reaction temperature. Solutions of reactant can be prepared by warming solutions to dissolve reactants, followed by mixing solutions and cooling to the desired reaction temperature. For example, supersaturated solutions of CBZ and NCT in ethanol are prepared by dissolving each reactant at 40 to 50° C. Solutions were then mixed at this temperature: 2.4 mL of 0.3 M CBZ solution and 3.6 mL of 1.4 M NCT solution and allowed to reach the reaction temperature, room temperature in this case. Crystallization did not occur during cooling. The concentration of the reactants in the solution is close to the solubility of each reactant at room temperature. The induction times for crystallization in ethanol were between 1 and 4 minutes. Similar behavior is observed in 2-propanol with crystallization times between 1 and 4 minutes and in ethyl acetate between 3 and 10 minutes. The method is also applied to the crystallization of carbamazepine:saccharin co-crystal, with induction times between 1 and 5 minutes in ethanol.

### Example 2a

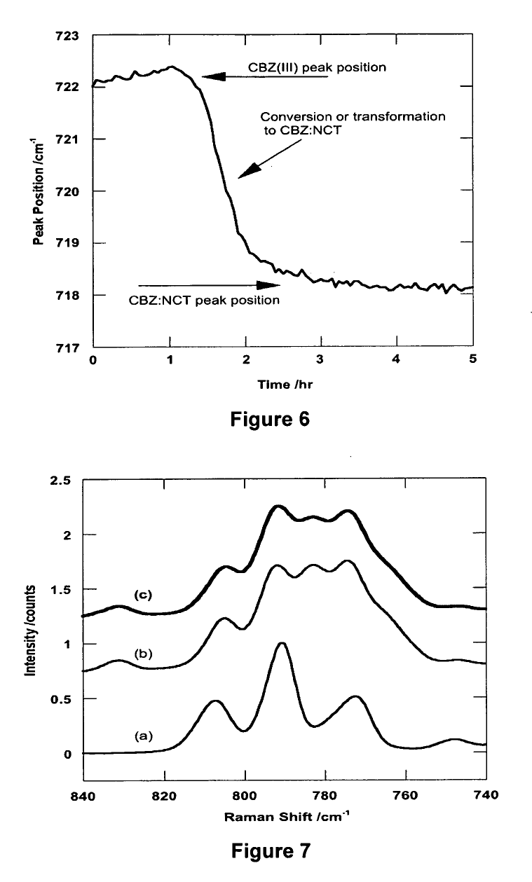

Carbamazepine: Nicotinamide co-crystal (CBZ:NCT) is obtained by adding solutions of NCT (about 0.5 M in 2-propanol) to solid anhydrous CBZ(III) (about 5-10 mg) at room temperature. Formation of co-crystal is confirmed by in situ-monitoring of the Raman peak at 718 cm⁻¹ in the precipitate in suspension, as discussed in Example 2b and FIG. 6.

### Example 2b

The CBZ:NCT co-crystal is also obtained by adding 10 mL of 0.16 M NCT ethanol solution to 385 mg anhydrous CBZ(III). FIG. 6 plot shows the shift in the Raman peak from 722 to 718 cm⁻¹ with respect to time during in-situ monitoring of the precipitation in suspension. The formation of the CBZ:NCT co-crystal in this system occurs between 1 and 3 hours.

A higher solution concentration of NCT in ethanol, 0.25 M, added to pure anhydrous CBZ(III) at room temperature, results in faster conversion to CBZ:NCT co-crystal, 2 to 3 minutes. This conversion is faster than the time needed to set up and collect the first Raman spectra.

### Example 2c

Carbamazepine:Nicotinamide co-crystal (CBZ:NCT) was obtained by adding an aqueous solution of NCT to solid dihydrate carbamazepine (CBZ(D)) at room temperature. The following Raman spectra (FIG. 7) shows that the initial solid phase of reactant, CBZ(D) transforms to CBZ:NCT co-crystal. Curve (a) is CBZ(D) reference; curve (b) is co-crystal reference; curve (c) is co-crystal of Example 2c.

### Example 2d

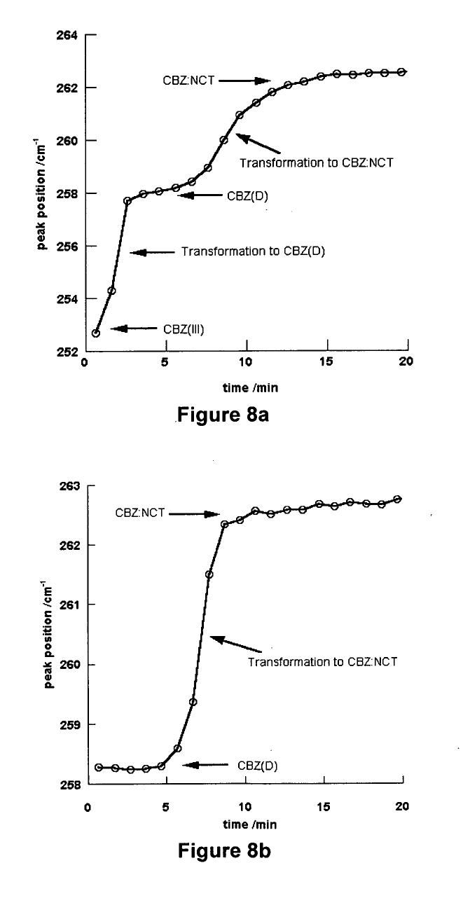

CBZ:NCT co-crystal is obtained by adding an 8M aqueous solution of NCT (2 mL) to solid CBZ(III) (500 mg) or to solid carbamazepine dihydrate (CBZ(D)) (500 mg) at room temperature. Raman spectra are collected for about 30 minutes after contacting the solid with the solution, to monitor the composition of the solids. In-situ monitoring of the suspension shows peak shifts from 253 cm⁻¹ to 263 cm⁻¹. FIG. 8 shows the Raman peak position with respect to time showing the slurry conversion of solid phase (FIG. 8a) CBZ(III) and (FIG. 8b) CBZ(D) to co-crystal CBZ:NCT at 23° C. Within this range, pure CBZ(III) has a peak center around 253 cm⁻¹. In water, it converts to dihydrate, which has a peak center shifted to 258 cm⁻¹. The experiments are stopped when the conversion to co-crystal is complete, indicated by a final Raman shift to 263 cm⁻¹.

### Example 3a

Carbamazepine:Nicotinamide co-crystal (CBZ:NCT) is prepared by adding a solution of one reactant to a suspension of the second reactant at room temperature. This method results in higher yields and shorter reaction times than those of Example 1. A first stream is prepared by adding 50 mg of CBZ(III): to a saturated ethanol solution of CBZ (3.75 mL of 0.1M). A second stream is an ethanol solution of NCT (2.25 mL of 0.8 M). The streams are combined and after initial gentle mixing, the system is left unstirred. Co-crystals are observed within 5 minutes. Similar results are observed by adding nicotinamide solid to a nicotinamide solution and adding a CBZ solution to the nicotinamide suspension.

### Example 3b

Carbamazepine:Nicotinamide co-crystal (CBZ:NCT) is obtained by adding 9 mL of 0.1 M NCT solution in ethyl acetate to a suspension of anhydrous CBZ(III) (390 mg) in ethyl acetate (10.18 g) at room temperature. One milliliter increments of solution are added every five minutes, so the transformation takes approximately 45 minutes. The vial is kept sealed with Parafilm™ after each addition of NCT solution. The Raman peak shifts from 722 cm⁻¹ to 718 cm⁻¹, indicating slurry conversion or transformation of CBZ(III) to CBZ:NCT co-crystal. The FTIR spectrum of the co-crystal is consistent with that of a CBZ:NCT reference.

### Example 3c

Sulfamethazine:2-Aminobenzoic Acid (SMZ:ANT) co-crystal is obtained by adding 6 mL of a saturated ANT/acetonitrile solution at room temperature to a suspension of solid SMZ (0.2098 g) in acetonitrile (8.1448 g). 2-Aminobenzoic acid is also known as anthranilic acid (ANT). The crystallized solids are those of the SMZ:ANT co-crystal. A Raman peak of sulfamethazine shifts from 992 cm⁻¹to 1010 cm⁻¹ upon addition of ANT solution, indicating slurry conversion or transformation of pure component crystal SMZ to SMZ:ANT co-crystal. The x-ray powder diffraction pattern of the co-crystal matches that of the SMZ:ANT reference.

Example 4a

Carbamazepine:Nicotinamide co-crystal (CBZ:NCT) is prepared in situ on a polarized and Raman microscope by adding a small drop of ethyl acetate, ethanol, or 2-propanol to the reactants in solid phase —NCT and CBZ(III)— on a depression slide at room temperature. The transformation from solid reactants to co-crystal is monitored by polarized light microscopy and by Raman microscopy. With ethyl acetate, the crystals grow in about 3 minutes and clearly show the crystalline needle growth of the CBZ:NCT co-crystal. Solid phase of the product is confirmed to be CBZ:NCT co-crystal by the spectra obtained by Raman microscopy.

### Example 4b

Carbamazepine:Nicotinamide co-crystal (CBZ:NCT) is prepared on a larger scale by adding 0.15 g water into a physical mixture of solid phase reactants NCT (0.122 g) and anhydrous CBZ(III) (0.236 g) at room temperature. The conversion of the initial solid reactants to CBZ:NCT co-crystal is complete within 60 minutes. Raman spectra from 3 random samples collected 60 minutes after mixing show the characteristic 718 cm⁻¹ Raman peak of the co-crystal.

### Example 4c

Carbamazepine:Saccharin co-crystal (CBZ:SAC) is prepared by adding 1.0254 g of 0.1 N HCl aqueous solution to a mixture of solid reactants CBZ(III) (0.2973 g) and SAC (0.2700 g) at room temperature. At the resulting pH, saccharin exists in solution in non-ionized form. The conversion of the initial solid reactants to CBZ:SAC co-crystal is confirmed from the FT-IR spectrum.

What is claimed is:

1. A method for precipitating a molecular complex from a solvent system, the molecular complex comprising two or more organic reactants held in complex by non-ionic interactions between the reactants, the method comprising adding a stoichiometric excess of one of the reactants to a solution comprising the two or more reactants.

2. A method according to claim 1, wherein the solution comprises the two or more reactants at stoichiometric levels corresponding to the presence of the reactants in the molecular complex.

3. A method according to claim 2, wherein the solution is saturated with respect to at least one of the reactants.

4. A method according to claim 1, comprising adding the stoichiometric excess as a solution.

5. A method according to claim 1, comprising adding the excess as a solid.

6. A method according to claim 1, comprising adding the excess in the form of a slurry.

7. A method according to claim 1, wherein the stoichiometric excess is at least 5:1.

8. A method according to claim 1, wherein the stoichiometric excess is at least 10:1.

9. A method according to claim 1, wherein at least one of the reactants is an active pharmaceutical ingredient.

10. A method according to claim 1, wherein the solvent comprises water.

11. A method according to claim 1, wherein the solvent comprises an organic liquid.

12. A method of producing a solid co-crystal composition by precipitation from a liquid phase, the co-crystal composition comprising a molecular complex of two or more organic reactants stabilized by non-ionic interactions between the reactants, the method comprising combining the reactants in a liquid solvent under over saturated conditions with respect to the molecular complex in the solvent,
wherein one reactant is present in the combined reactants and solvent in a molar excess of at least 2:1, relative to its presence in the co-crystal.

13. A method according to claim 12, wherein the combination contains at least a 5:1 molar excess of one of the reactants.

14. A method according to claim 12, wherein the combination contains at least a 10:1 molar excess of one of the reactants.

15. A method according to claim 12, comprising combining solutions of the reactants.

16. A method according to claim 12, comprising adding one of the reactants in solid form to a solution comprising the other reactant or reactants.

17. A method according to claim 12, comprising adding a solution of one reactant to a suspension comprising the other reactant or reactants.

18. A method according to claim 12, comprising adding solvent to solid reactants.

19. A method according to claim 12, wherein at least one of the reactants is an active pharmaceutical ingredient.

20. A method for making pharmaceutical co-crystals by precipitation of a solid form from a solvent, wherein the pharmaceutical co-crystal is a solid molecular complex between two or more reactants where the reactants are held in the complex by non-ionic interactions, and wherein one of the reactants is an active pharmaceutical ingredient, the method comprising combining the active pharmaceutical ingredient, other reactant, and solvent under over saturation conditions with respect to the complex in the solution,
wherein one of the active pharmaceutical ingredient and the other reactant or reactants is provided in a molar excess of at least 2:1 with respect to its presence in the complex.

21. A method according to claim 20, wherein the molar excess is at least 5:1.

22. A method according to claim 20, wherein the molar excess is at least 10:1.

23. A method according to claim 20, wherein the active pharmaceutical ingredient is provided in molar excess.

24. A method according to claim 20, wherein a reactant other than the active pharmaceutical ingredient is provided in molar excess.

25. A method according to claim 20, wherein the active pharmaceutical ingredient is selected from the group consisting of carbamazepine, caffeine, itraconazole, fluoxetine hydrochloride, aspirin, ibuprofen, flurbiprofen, sulfamethazine, sulfamethoxypyridazine, sulfametrole, sulfamethoxazole, theophylline, and mebandazole.

26. A method of screening for the formation of a non-ionic molecular complex from prospective reactants, comprising
combining the prospective reactants in a solvent, wherein one of the prospective reactants is present in a molar excess of at least 2:1 with respect to the presence of the prospective reactants in the co-crystal; and
if a precipitate forms, analyzing the precipitate to confirm or determine that it is a co-crystal form comprising the reactants.

27. A method according to claim 26, comprising measuring a Raman band of the precipitate.

28. A method according to claim 26, comprising analyzing the precipitate by x-ray diffraction.

29. A method according to claim 26, comprising analyzing the precipitate by infrared spectroscopy.

30. A method according to claim 26, wherein combining the prospective reactants comprises adding together separate solutions of the reactants.

31. A method according to claim 29, wherein the reactants have different solubilities in the solvent and the solution of the reactant provided at the lower molar ratio is saturated.

32. A method according to claim 26, wherein combining the perspective reactants comprises adding one reactant as a solid to a solution of another reactant.

33. A method according to claim 26, carried out combinatorially.

34. A method for decreasing the solubility of a molecular complex in a solvent system, the molecular complex comprising two or more organic reactants in complex held together by non-ionic interactions between the reactants, the method comprising adding a stoichiometric excess of one of the reactants to a solution comprising the complex.
